(19) United States
(12) Patent Application Publication
(10) Pub. No.: US 2014/0079745 A1
(43) Pub. Date: Mar. 20, 2014

(54) COSMETIC COMPOSITIONS COMPRISING POWDER CONTAINING ALUMINUM HYDROXIDE

(71) Applicants: Katsumi SHIMIZU, Pomfret Center, CT (US); Shoji Takekawa, Shrewsbury, MA (US)

(72) Inventors: Katsumi SHIMIZU, Pomfret Center, CT (US); Shoji Takekawa, Shrewsbury, MA (US)

(73) Assignee: U.S. Cosmetics Corporation, Dayville, CT (US)

(21) Appl. No.: 13/622,860

(22) Filed: Sep. 19, 2012

(51) Int. Cl. A61K 8/02; A61Q 1/02; A61Q 1/06; A61Q 1/04; A61Q 1/08; A61K 33/08; A61Q 1/10; A61Q 17/04; A61Q 15/00

(52) U.S. Cl. 424/401; 424/59; 424/63; 424/64; 424/65; 424/70.7; 424/690

(57) ABSTRACT

The embodiments relate to cosmetic compositions providing a high coverage to the skin while retaining a natural skin appearance. The compositions of embodiments include a powder-containing aluminum hydroxide particles having an average coated ratio of from about 60% to about 150% powder by weight of aluminum hydroxide, and a cosmetically acceptable carrier. The compositions may be useful as a foundation and/or as a composition to correct skin discoloration.

### FIELD OF THE INVENTION

The embodiments relate to cosmetic compositions providing a high coverage to the skin while retaining a natural skin appearance, in which the compositions include a powder-containing aluminum hydroxide particles and a cosmetically acceptable carrier. The compositions may be useful as a foundation and/or as a composition to correct skin discoloration, and may be used to cover skin imperfections while at the same time retaining a natural skin appearance.

### BACKGROUND

Cosmetic compositions are used by consumers for regulating the condition of the skin and/or for improving the appearance of the skin. Cosmetic compositions, such as foundations, are popular amongst consumers, because they are capable of masking skin imperfections and skin tonal, variations—this ability is referred to as “coverage.” These compositions also may provide coloration to the skin by incorporating conventional metal oxide pigments into the compositions, such as iron oxide pigments and titanium dioxide and other powder. Cosmetic compositions also are popular amongst consumers, because they are capable of correcting skin discoloration.

Depending on the consumers' taste and/or the degree of skin imperfections and/or the skin tonal variations and/or the skin discoloration surrounding the eyes, a high coverage effect may be desired and/or needed. A high coverage effect may be obtained by incorporating a high proportion of pigments, especially pigmentary grade titanium dioxide particles, into the cosmetic compositions. Application onto the skin of a composition providing high coverage, however, usually impairs the natural appearance of the skin. In particular, the application onto the skin of a composition providing high coverage usually results in a white hue across the skin. The white hue is also known as “chalkiness” for consumers having darker skins and “pastiness” for consumers having lighter skins. There is a need, therefore, to provide a cosmetic composition providing high coverage, particularly for masking skin imperfections and/or tonal variations of the skin, while retaining a natural skin appearance. There also is a need to provide a cosmetic composition correcting the skin discoloration surrounding the eyes, while retaining a natural skin appearance. There also is a need to provide a cosmetic composition which delivers high coverage whilst imparting a minimal white hue (ghost-like, doll-like effect) to the skin, or even whilst imparting no white hue to the skin. There also is a need to provide a cosmetic composition which delivers high coverage to darker skin whilst imparting minimal chalkiness, or even whilst imparting no chalkiness. There also is a need to provide a cosmetic composition which delivers high coverage to lighter skin whilst imparting minimal pastiness, or even whilst imparting no pastiness. Finally, there is a need to provide a cosmetic composition that delivers high coverage to the skin, and that minimizes further the white hue to the skin, compared to currently marketed cosmetic compositions.

Chalkiness and/or pastiness, i.e. the white hue across the skin, and more generally an unnatural appearance of the skin, is generally associated with the reflectance angle of light. When using conventional titanium dioxide, there is a specular reflection observed. This undesirable effect may be reduced by incorporating a lower proportion of pigmentary grade titanium dioxide particles into the cosmetic compositions, but doing that has the disadvantage of reducing the coverage provided to the skin. There is a need, therefore, to provide a cosmetic composition that provides high coverage of skin while minimizing the specular reflection.

The use of aluminum hydroxide powder in cosmetic compositions is known. Typically, the compositions include aluminum hydroxide deposited on the surface of a powder as a coating, as described for example, in U.S. Pat. Nos. 4,084,983, 4,323,554, 4,309,480, 4,968,351, 5,156,889, 6,416,573, 6,531,524, 6,630,019, 7,531,184, and U.S. Patent Application Publication No. 2001/0005735, the disclosures of each of which are incorporated by reference herein in their entireties. It also has been known to use alumina, not aluminum hydroxide, as a powder particle, and coat the alumina at least partially with titanium dioxide (JP 2008-088317), or phosphoric acid or phosphate (U.S. Pat. No. 6,015,456). It also has been described in U.S. Pat. No. 7,682,604 to provide a composite powder useful in cosmetic compositions in which a flaky substrate powder (e.g., mica) is contacted with a seed particle that acts as nuclei on the surface of the flaky substrate to grow barium sulfate particles or zinc oxide particles. Seed particles include titanium oxide, zinc oxide, alumina, aluminum hydroxide, silica, and iron oxide.

Accordingly, what is needed in the art is a pigment that can be used in cosmetic systems without using a significant amount of emulsifiers.

### BRIEF SUMMARY

According to a first aspect, an embodiment relates to a cosmetic composition comprising powder-containing aluminum hydroxide particles having an average coated ratio from about 60% to about 150% powder by weight of the aluminum hydroxide and, a cosmetically acceptable carrier. The aluminum hydroxide particles serve as the base and coated with, or have contained on the surface thereof, a powder. This composition may be suitable for providing high coverage while minimizing the specular reflection.

According to another feature of an embodiment, there is provided a method of making powder-containing aluminum hydroxide particles that comprises preparing an aqueous sodium hydroxide (NaOH aq) solution, adding a powder to the solution with high speed to homogenize the mixture to a homogenized powder mixture that is uniformly dispersed. The homogenized powder mixture then is contacted with an aqueous aluminum sulfate (Al₂(SO₄)₃) solution with high speed. The method further includes filtering and drying the powder to produce an aluminum hydroxide coated powder.

According to another feature of an embodiment, there is provided a powder-containing aluminum hydroxide particles having an average coated ratio from 60% to 150% powder by weight of the aluminum hydroxide for use as foundations in cosmetic compositions. Specifically, the powder-containing aluminum hydroxide particles are present in an amount of from about 5% to about 50%, by weight of the composition.

### BRIEF DESCRIPTION OF THE DRAWINGS

FIG. 1 is a comparison of SEM micrographs of the powder prepared in accordance with Example 1, and the powder prepared in accordance with U.S. Pat. No. 6,416,573.

FIG. 2 illustrates the mechanism by which a liquid formulation containing the powder prepared in accordance with Example 1 was compared to a control, and to Bioskin itself using the CIE color comparison technique.

FIG. 3 is a graph showing the reflection strength of the powder prepared in accordance with Example 1.

### DETAILED DESCRIPTION OF THE INVENTION

This invention relates to a powder that comprises aluminum hydroxide as the substrate coated with a powder, in which the aluminum hydroxide substrate has an average coated ratio from 60% to 150% powder by weight of the aluminum hydroxide particles. The powder is useful in cosmetic formulations together with a cosmetically acceptable carrier. While it is possible to use the powder in cosmetic formulations, and especially as a foundation formulation, the powder also may be useful in inks, paints, varnishes, toners, sunscreen formulations, and the like.

The following definitions and non-limiting guidelines must be considered in reviewing the description of this invention set forth herein. The headings (such as “Background” and “Brief Summary,”) and sub-headings used herein are intended only for general organization of topics within the disclosure of the invention, and are not intended to limit the disclosure of the invention or any aspect thereof. In particular, subject matter disclosed in the “Background” may include aspects of technology within the scope of the invention, and may not constitute a recitation of prior art. Subject matter disclosed in the “Brief Summary” is not an exhaustive or complete disclosure of the entire scope of the invention or any embodiments thereof. Classification or discussion of a material within a section of this specification as having a particular utility (e.g., as being an “active” or a “carrier” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. Any discussion of the content of references cited in the Background is intended merely to provide a general summary of assertions made by the authors of the references, and does not constitute an admission as to the accuracy of the content of such references.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features. Examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention. In addition, the compositions and the methods may comprise, consist essentially of, or consist of the elements described therein.

As used throughout, ranges are used as a shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Throughout this description, the use of the term “about” or “approximately” is intended to denote an approximation of the number, which includes the number modified by the term, and a reasonable deviation from that term, including standard measurement errors. Unless otherwise specified, all percentages and amounts expressed herein and elsewhere in the specification should be understood to refer to percentages by weight. The amounts given are based on the active weight of the material. The recitation of a specific value herein is intended to denote that value, plus or minus a degree of variability to account for errors in measurements. For example, an amount of 10% or about 10% may include 9.5% or 10.5%, given the degree of error in measurement that will be appreciated and understood by those having ordinary skill in the art.

As used herein, the term “cosmetic composition” means a composition which is intended to be applied onto the consumer's skin, particularly onto the facial skin or onto the body skin area or onto hair, so as to regulate the condition of the skin and/or to improve the appearance of the skin. The term “powder” means any insoluble material which particle size is 0.01 micrometer to 50 micrometers used for cosmetics. The term “average primary particle size” of powder-containing aluminum hydroxide particles means the equivalent volume mean primary particle size of the elementary powder-containing aluminum hydroxide crystals. The average primary particle size is measured on the powder-containing aluminum hydroxide, before being coated.

Throughout this description, the term “foundation” means a cosmetic composition which is intended to be applied onto the consumer's skin, particularly, onto the facial skin, body skin and hair so as to provide coverage and/or to mask skin irregularities and/or skin imperfections and/or skin tonal variations. The term “chalkiness” means the white hue which is observed onto skin after applying onto skin, particularly darker skin, a cosmetic composition providing high coverage. The term “pastiness” means the white hue that may be observed on the skin after applying onto skin, particularly lighter skin, a cosmetic composition providing high coverage.

All percentages, ratios and proportions herein are by weight, unless otherwise specified. All such weights as they pertain to listed ingredients are based on the active level, unless otherwise specified.

Embodiments of the invention include cosmetic compositions that contain powder-containing aluminum hydroxide particle substrates coated with a powder in which the coating is present in an amount of from about 60% to 150% of coating by weight of the aluminum hydroxide. In one embodiment, the powder is present in an amount of from about 75% to about 125% of powder by weight of the aluminum hydroxide, and in another embodiment, the powder is present in an amount of about 100% of powder by weight of the aluminum hydroxide.

The powder that forms the coating on the aluminum hydroxide particle substrate can be any powder useful in cosmetic compositions including those that provide one or more of the following effects: luster effect, oil absorption, feeling improvement, cover effect, and the like. The particular powder used is not critical, although the embodiments provide a powder capable of forming a coating on the aluminum hydroxide substrate in amounts within the coating ratios described herein. Suitable powders include, for example, those belonging to the clay mineral group: such as those containing illite groups such as sericite (silky mica), muscovite, biotite, lithia mica, and synthetic mica; those containing kaolin groups such as kaolionite, nacrite, dekkite, halloysite; those containing sillimanite groups such as sillimanite and kyanite; magnesium silicate systems such as talc, and serpentine groups; and titanium dioxide and zinc dioxide. A single powder may be used to coat the aluminum hydroxide substrate, or combinations of powders may be used to specifically tailor the powder-containing aluminum hydroxide.

The average particle size of the powder that forms the coating on the aluminum hydroxide particles can be within the range of from about 0.01 to about 75 μm, more preferably from about 0.05 to about 65, even more preferably from about 0.1 to about 50 μm, and most preferably from about 1 to about 25 μm. The average thickness of the powder may be within the range of from about 0.01 to about 3 μm, more preferably from about 0.1 to about 2, and most preferably from about 0.1 to about 1.5 μm. In an embodiment, the powder material may have a refractive index within the range of from about 1.25 to about 1.95, more preferably from about 1.40 to about 1.80, and most preferably from about 1.45.about.1.65.

The refractive index of oil solutions that are usually used in cosmetics typically is within the range of from about 1.39 to about 1.51, while the refractive index of mica is 1.59 and talc is 1.53, and the refractive index of the stratum corneum is 1.55. As can be seen from the numbers above, when normal pigments are used in cosmetics, the refractive indicies are very similar to each other, and when the powders are wetted with skin secreted sebum at its oil absorption or over this amount, the powders that make up the cosmetic film on the skin become transparent. When an excess amount of sebum is secreted, a reflection from the surface of the sebum film, as well as the reflection from the surface of the powder dispersed within the sebum, will emphasize and create an undesirable shine, that when viewed from different angles will make the wrinkles stand out, and in occasion make the wearer of the cosmetic have a very tired look. By forming the aluminum hydroxide substrate coated with the powder in accordance with the embodiments, the shine can be reduced significantly, and the resulting powder has improved specular reflection and reflection strength.

The final powder comprised of the aluminum hydroxide particles coated with the powder may have an average particle size within the range of from about 0.01 to about 50 μm, and in some embodiments the average particle size can be within the range of from about 0.1 to about 45 μm, and in other embodiments, the average particle size can be within the range of from about 1 to about 35 μm. The powder can be useful in a cosmetic composition to provide a high coverage of skin while retaining a natural skin appearance, and can be used in such a composition in an amount of from about 1 to about 65% by weight. In other embodiments, the powder can be used in an amount of from about 3 to about 55% by weight, in other embodiments from about 5 to about 50% by weight, and in yet other embodiments, from about 10 to about 30% by weight.

Depending on the formulation (e.g., liquid formulation, powder formulation, skin lotion, body soap, etc.), the amount of the powder can vary widely. For example, for a liquid formulation, the amount of the aluminum hydroxide coated powder can be used in an amount of from about 5 to about 50% by weight, or from about 10 to about 35% by weight, or from about 15 to about 25% by weight, or at about 20% by weight. For a powder formulation, such as makeup foundation or the like, the amount of the aluminum hydroxide coated powder can be used in an amount of from about 5 to about 50% by weight, or from about 15 to about 40% by weight, or from about 25 to about 35% by weight, or at about 20% by weight. For a skin lotion formulation, the amount of the aluminum hydroxide coated powder can be used in an amount of from about 1 to about 30% by weight, or from about 3 to about 20% by weight, or from about 4 to about 10% by weight, or at about 5% by weight. For a body soap formulation, the amount of the aluminum hydroxide coated powder can be used in an amount of from about 2 to about 40% by weight, or from about 5 to about 20% by weight, or from about 7 to about 15% by weight, or at about 10% by weight.

The cosmetic compositions useful in the embodiments described herein also may contain other conventional components useful in various cosmetic compositions. Any cosmetically acceptable vehicle may be used together with the aluminum hydroxide powder. Such vehicles may include, for example, water, glycerin, dimethicone, beeswax, glyceryl stearate, and the like. Other ingredients normally used in cosmetics also may be present, when desired. For example, inorganic powders such as talc, kaolin, sericite, muscovite, phlogopite, red mica, biotite, synthetic mica, lithia mica, vermiculite, magnesium carbonate, calcium carbonate, diatomite, magnesium silicate, calcium silicate, aluminum silicate, barium silicate, barium sulfate, strontium silicate, wolframic acid metal salt, or silica, hydroxyapatite, zeolite, boron nitride, ceramic powder, organic powders such as nylon powder, polyethylene powder, polystyrene powder, benzoguanamine powder, polyfluoridation ethylene powder, di-styrene benzene polymer powder, epoxy powder, acrylic powder, silicone powder, microcrystalline cellulose, inorganic white pigments such as titanium dioxide and zinc oxide, inorganic red system pigments such as iron oxide (red iron oxide) and titanic acid irons, inorganic brown system pigments such as γ-iron oxides, inorganic yellow system pigments such as yellow soil and yellow iron oxides, inorganic black color system pigments such as tetravalent acid iron oxide, carbon black, inorganic violet system pigments such as mango violet, cobalt violet, inorganic green system pigments such as chromium oxide, chromium hydroxide, and titanic acid cobalt, inorganic blue system pigments such as ultramarine blue, and prussian blue, pearl pigments such as titanium dioxide covered mica, titanium dioxide covered bismuth oxychloride, bismuth oxychloride, titanium dioxide covered talc, fish scale foil, colored titanium dioxide covered mica, metal powder pigment such as aluminum powder, copper powder, colored composite pigments such as iron-doped zinc oxide and iron-doped titanium dioxide.

Other pigments may be used, such as red No. 201, red No. 202, red No. 204, red No. 205, red No. 220, red No. 226, red No. 228, red No. 405, orange-colored No. 203, orange-colored No. 204, yellow No. 205, yellow No. 401 and blue No. 404, organic chlorophyll pigment such as FD&C Red No. 3, red No. 104, red No. 106, red No. 227, red No. 230, red No. 401, red No. 505, orange-colored No. 205, FD&C Yellow No. 4, yellow No. 5, yellow No. 202, yellow No. 203, orange-colored No. 3 and zirconium, barium, or aluminum lake of blue No. 1, natural colorants such as β-carotene, hydrocarbon oils such as squalane, mineral oil, vaseline, micro crystalline wax, ozokerite, ceresin, myristic acid, palmitic acid, stearic acid, oleic acid, iso-stearic acid, cetyl alcohol, hexadecyl alcohol, oleyl alcohol, cetyl 2-ethylhexanoate, 2-ethylhexyl palmitate, 2-octyldodecyl myristate, neo-pentylglycol di-2-ethylhexanoate, glyceryl tri-2-ethylhexanoate, 2-octyldocyl oleate, isopropyl myristate, glyceryl triisostearate, caprylic/capric triglyceride, olive oil, avocado oil, yellow bees wax, myristyl myristate, mink oil, lanolin oil, silicone oil, higher fatty acid oil, ester oils of fatty acids, higher alcohol, oil components of wax groups, cyclopentasiloxanes, dimethicones, trimethylsiloxysilicates, and organic solvents such as acetone, toluene, butyl acetate, and ester acetate can be used in various amounts.

Resins such as alkyd resin, urea-formaldehyde resin, Nylon-12, plasticizers such as camphor, acetyl tributyl citric acid, ultraviolet absorbing agents, antioxidants, antiseptics, surfactants, moisturizing agents, perfumes, water, alcohol, and thickeners can also be used.

The powder aluminum hydroxide coated with a powder can be prepared by preparing an aqueous sodium hydroxide (NaOH aq) solution, adding a powder to the solution with high speed to homogenize the mixture to a homogenized powder mixture that is uniformly dispersed. The homogenized powder mixture then is contacted with an aqueous aluminum sulfate (Al₂(SO₄)₃) solution with high speed. The method further includes filtering and drying the powder to produce an aluminum hydroxide coated powder.

In one embodiment the aqueous sodium hydroxide solution and the aqueous aluminum sulfate solutions are prepared as pre-mixes and then carry out the method by first adding a powder to the aqueous sodium hydroxide pre-mix. The pre-mix with the added powder then is homogenized in a stirred reactor, or homogenizer apparatus using any conventional apparatus to homogenize the mixture. In one embodiment, the mixture is homogenized and the temperature is increased to within a range of from about 30 to about 75° C., or within a range of from about 40 to about 60° C., or to about 50° C. After the mixture is homogenized and the temperature achieved, the aqueous aluminum sulfate pre-mix can be added with continuous homogenization. In one embodiment, the aqueous aluminum sulfate pre-mix is added step-wise using any mechanism capable of metering in the solution (e.g., pump, valve, titration, etc.) until the pH of the mixture reaches a value within the range of from about 5.5 to about 8.5, or until the pH of the mixture reaches a value within the range of from about 6.5 to about 7.5, or until the pH of the mixture reaches about 7.0.

The resulting mixture will contain aluminum hydroxide substrate particles, coated with the powder. The particles then can be separated from the mixture using any known mechanism capable of filtering the particles from the mixture, including for example, conventional filter paper, filter press, or other known filtering apparatus. The particles separated by filtration then can be dried using any known mechanism capable of drying particles, including kiln or oven drying, passing the particles through a heated oven or press, spray drying, and the like. Drying can take place for a period of time from about 1 hour to 30 hours, or from about 5 hours to about 20 hours, or from about 12 hours to 18 hours, or about 16 hours, depending on the drying temperature. The drying temperature may be within the range of from about 75° C. to about 175° C., or from about 90° C. to about 120° C., or about 105° C. The final dried particles comprise aluminum hydroxide powder substrates, coated with the powder.

In one embodiment, water and sodium hydroxide may be mixed to form a first pre-mix, in which the amount of aqueous sodium hydroxide solution is from about 125 to about 185% by weight based on the weight of the powder, or from about 145 to about 175% by weight based on the weight of the powder, or from about 160% by weight based on the weight of the powder. Water and aluminum sulfate also may be mixed to form a second pre-mix, in which the amount of aqueous sodium hydroxide solution is from about 185 to about 225% by weight based on the weight of the powder, or from about 195 to about 215% by weight based on the weight of the powder, or from about 200% by weight, based on the weight of the powder.

In accordance with an embodiment, a powder, such as titanium dioxide, kaolin, mica, talc, or the like may be added to the first pre-mix, and the resulting mixture homogenized and the temperature increased as stated above. The second pre-mix then may be added to the homogenized mixture containing the powder in a step-wise manner until the pH reaches a value as described previously. The resulting particles then can be filtered and dried as discussed above.

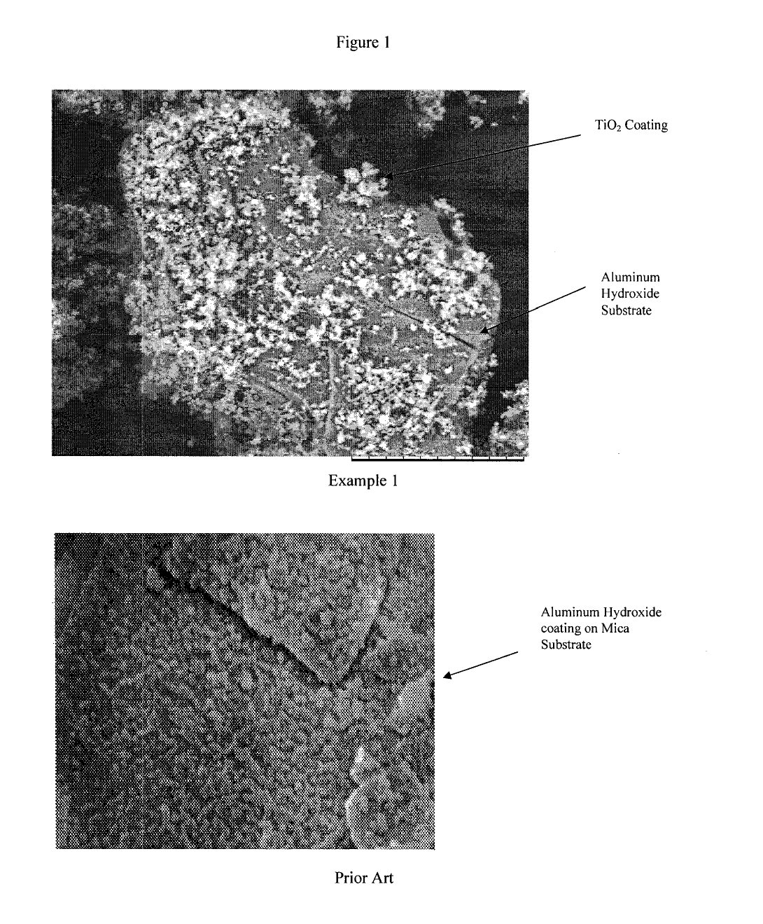

The method can be carried out in a batch or continuous process to produce the powder particles comprised of an aluminum hydroxide substrate coated with a powder in an amount of from about 60% to 150% of powder coating by weight of the aluminum hydroxide. The term “coating” or “coated” is not meant to denote a uniform distribution of powder over the entire surface. Rather, as seen in FIG. 1, the particles of powder adhere to the surface of the aluminum hydroxide substrate and do not necessarily coat the entire surface, although in some embodiments, the powder can be adhered over the entire surface of the aluminum hydroxide substrate. In some embodiments, the powder adheres to about 10 to about 95% of the surface, or from about 20 to about 80%, or from about 30 to about 70%, or from about 40 to about 65% of the surface of the aluminum hydroxide.

The aluminum hydroxide particles that are coated with the powder can be used in cosmetic compositions, together with a cosmetically acceptable carrier. The formulation of the particular cosmetic formulation, or other product, can be carried out using conventional techniques. The inventors have discovered that the aluminum hydroxide particles have strong back-scattering properties, rendering them particularly suitable for foundations, (liquid and powder), skin lotions, body soaps, and other similar cosmetic compositions. The aluminum hydroxide particles may have a specular reflection, when measured using a Spectrophotogoniometer (GC5000L, commercially available from Nippon Denshoku Industries Co., Ltd,), at 0°/45° of from about 95% to about 145%, or from about 100% to about 125%, or from about 110 to about 115%. The particles also have a reflection strength, when measured using a Spectrophotogoniometer (GC5000L, commercially available from Nippon Denshoku Industries Co., Ltd,) of less than 100%, at a ratio of 0°/45°. The sum total of each angle reflection strength should be equal to incident light strength, which means that each angle reflection strength should be less than 100%. The reflection strength is important in confirming that the sample has reflection characteristics.

The aluminum hydroxide particles having improved back-scattering properties, can be used in foundation formulations so that the resulting foundation matches the skin color. In some embodiments, the difference in color between the skin and the foundation formulation on the skin, is not noticeable to the human eye, when measured using the CIE LAB color scale. In embodiments, the CIE LAB color scale measurement (ΔE) is within the range of from about 0.1 to about 4.5, or from about 0.5 to about 2.0, or from about 0.9 to about 1.25, or about 0.97, when measured on Bioskin.

While the aluminum hydroxide particles are particularly useful in cosmetic compositions, there are no restrictions to the form of cosmetics in which the particles can be used. For example the particles can be used in several forms such as powder form, cake form, pencil and stick form, pellet form, ointment form, liquid form, milky lotion form, or cream form. The particles, especially when using titanium dioxide, mica, talc, or kaolin as the powder material, is very useful in cosmetic formulations, but can also be used in various other fields besides cosmetics, such as for industrial purposes, such as inks, paints (coating), plastics, rubber additives, rubber moldings, rubber mold separating material etc., and other various lubricants.

The aluminum hydroxide powder particles may be used in cosmetic products, such as foundations, lip sticks, eye shadow, lotions, creams, concealer, blush, eyeliners, mascara, eyebrow liner, lipliner, and sunscreen. They may also be used in toiletry products, such as deodorants, antiperspirants, body soaps, and shower gels. When the particles are used in a cosmetic product or a toiletry product, other typical components used in making the cosmetic product or toiletry product can be added to the powder-containing aluminum oxide. For instance, lip stick will often contain various oils and waxes in addition to the pigments. The powder particles may be used with other powders, pigments, or other components, including the same powder adhered to the aluminum hydroxide substrate, but added separately.

The following examples are intended to illustrate the invention. These examples should not be used to limit the scope of the invention, which is defined by the claims.

### Example 1

### Preparation of Aluminum Hydroxide Particle Coated with TiO² Powder

An aqueous sodium hydroxide first pre-mix solution was prepared by mixing water and sodium hydroxide to form a first pre-mix in an amount of 160% of the amount of titanium dioxide powder added. An aqueous aluminum sulfate second pre-mix solution was prepared by mixing water and aluminum sulfate in an amount of 200% of the amount of titanium dioxide powder added. Titanium dioxide powder was added to the first pre-mix, the mixture was homogenized in a high speed mixer, and the temperature was raised to 50° C. The second pre-mix then was added step-wise with a controlled pump to the homogenized and heated mixture containing the titanium dioxide powder until the pH of the mixture reached 7.0. The resulting mixture then was filtered using a paper filter and the filter cake was dried in an oven for 16 hours at a temperature of 105° C. to prepare an aluminum hydroxide powder coated with titanium dioxide.

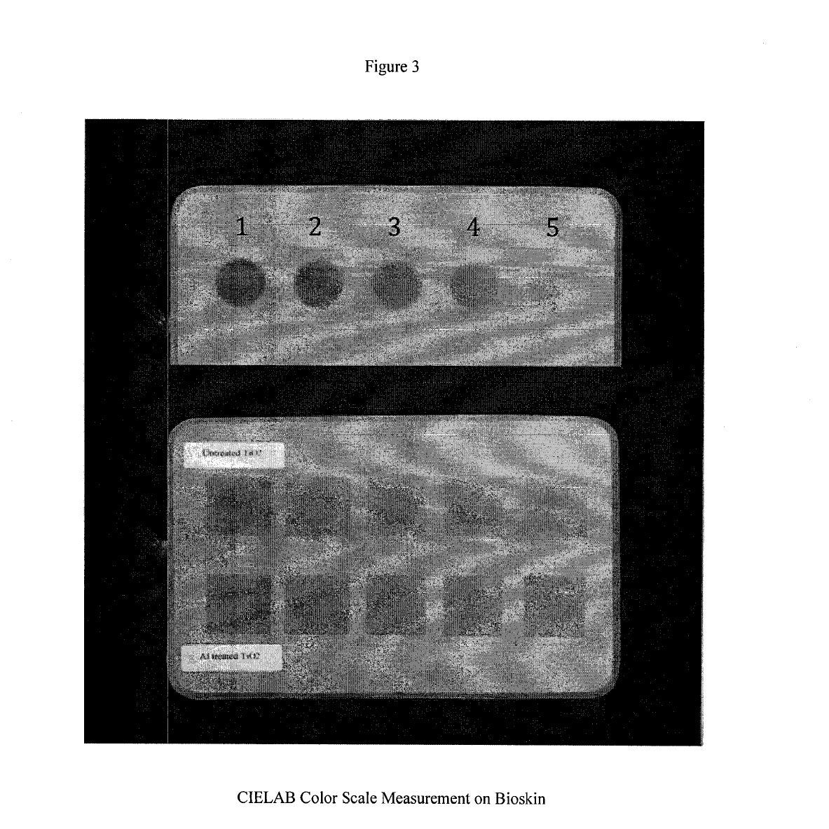

An SEM of the aluminum hydroxide powder coated with titanium dioxide was taken and is shown in the upper portion of FIG. 1. FIG. 3 of U.S. Pat. No. 6,416,573 (the “'573 patent”) is shown in the lower portion of FIG. 1 for comparison purposes. FIG. 3 of the '573 patent shows a mica substrate coated with aluminum hydroxide, whereas the powder prepared in accordance with Example 1 shows an aluminum hydroxide substrate coated with titanium dioxide powder particles. Thus, the aluminum hydroxide of the present embodiments forms the substrate of the powder, whereas aluminum hydroxide in the '573 patent forms the coating on a powder substrate such as mica.

### Example 2

### Measurement of Reflective Properties

The aluminum hydroxide powder of Example 1 was compared to controls, and conventional powder materials using a Spectrophotogoniometer (GC5000L, commercially available from Nippon Denshoku Industries Co., Ltd,). The samples were measured at 0° incident light, and at 45° incident light, and the differences, or % change, are reflected in the Table below. Each sample (1.0 mg) was placed on a 5 cm×5 cm sample of Bioskin, and measured at 0° and 45° specular reflection.

TABLE 1
VSpecular Reflection

| Sample | 0°/45° |
|---|---|
| Control (TiO₂ only) | 30.21% |
| Mica | 41.45% |
| Example 1 | 111.36% |
| CR-50 (TiO₂) | 75.94% |
| Ultra Fine TiO₂ | 44.10% |
| KOSE-1 | 89.37% |
| KOSE-2 | 71.29% |
| KOSE-3 | 74/93% |

KOSE-1, KOSE-2, and KOSE-3 are samples from Japanese Patent Application JP2008-88317

As seen from the above table, the inventive sample provided a far greater degree of back-scattering when compared to known powders. Generally, a value close 100% means that the sample has soft focus, whereas values over 100% mean that the sample has back scattering.

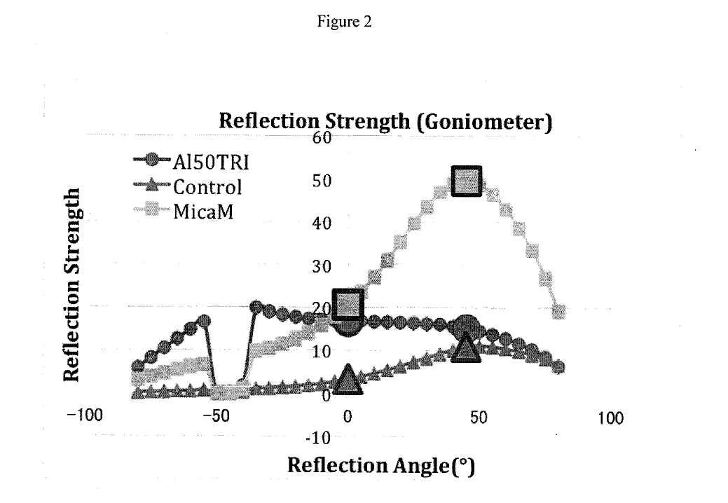

Using the same Spectrophotogoniometer, the powder of Example 1 (labeled AL50TRI in FIG. 2) was compared to conventional powders, including a control (TiO2), and mica, and the reflection strength was plotted against the reflection angle. The resulting curves are shown in FIG. 2. Typically, the reflection is almost forward scattering, but multi direction scattering is important for making soft focus. The powder prepared in accordance with the present embodiments have back scattering, as shown by the multi-scattering of the inventive sample in FIG. 2.

### Example 3

### Liquid Foundation Formulation

The aluminum hydroxide particles prepared in accordance with Example 1 were used in a liquid foundation formulation having the following components:

| Ingredient | Amount |
|---|---|
| Example 1 particles | 20.0% |
| Nylon-12 | 2.0% |
| Talc | 2.0% |
| Mica | 1.0% |
| Iron Oxide | 1.0% |
| Isononyl Isononate | 9.0% |
| Cyclopentasiloxane | 8.5% |
| PEG-9 Dimethicone | 1.5% |
| Water | 45.0% |
| Glycerin | 10.0% |

The liquid foundation formulation prepared in accordance with Example 3 was then compared to an identical liquid foundation formulation with the exception that the Example 1 particles were replaced with titanium dioxide powder. These foundation formulations were applied to Bioskin, commercially available from Beaulax Co., Ltd. (http://www.bioskin.ne.jp/english/index.html). Various age spots having a variety of darknesses were applied to the Bioskin (numbered 1-5), as shown in FIG. 3. To each age spot was applied the inventive formulation and the comparative example, and a control was prepared by not applying any foundation formulation. Each spot then was subjected to a CIE LAB color evaluation and the difference between the foundation area and the age spot area were calculated using the conventional L-a-b method (where L represents brightness or white, a represents the degree of redness, and b represents the degree of yellowness). The ΔE between the foundation area (or Bioskin in the case of the control) and the age spot were measured for each of the 5 age spots, and the values are reported in Table 2 below.

TABLE 2
Color Evaluation Results

| | ΔE | | | | |
|---|---|---|---|---|---|
| | 1 | 2 | 3 | 4 | 5 |
| Control | 15.16 | 14.61 | 13.72 | 11.31 | 4.69 |
| (Bioskin) | | | | | |
| TiO₂ | 5.81 | 4.69 | 4.44 | 3.79 | 1.45 |
| formulation | | | | | |
| Example 3 | 4.55 | 3.32 | 2.52 | 1.77 | 0.97 |

The results from the CIE LAB color evaluation reveal that the liquid foundation prepared in accordance with Example 3 provided superior coverage when compared to the control and the comparative formulation. In most instances the inventive example provided an improved color match, when compared to the control, of from 50% to about 90%, or from about 60% to about 80% (control value/example 3). When compared to the comparative formulation, the inventive example provided an improved color match of from about 20% to about 60%, or from about 25% to about 50%.

### Example 4

### Powder Foundation Formulation

The aluminum hydroxide particles prepared in accordance with Example 1, except mica was used in place of TiO₂, were used in a powder foundation formulation having the following components:

| Ingredient | Amount |
|---|---|
| Aluminum hydroxide with Mica | 30.0% |
| Nylon-12 | 5.0% |
| Talc | 25.0% |
| Titanium Dioxide | 20.0% |
| Iron Oxide | 1.0% |
| Trimethylsiloxysilicate | 1.0% |
| Cyclopentasiloxane | 7.5% |
| Dimethicone | 5.5% |

### Example 5

### Skin Lotion Formulation

The aluminum hydroxide particles prepared in accordance with Example 1, except talc was used in place of TiO₂, were used in a skin lotion formulation having the following components:

| Ingredient | Amount |
|---|---|
| Aluminum hydroxide with talc | 5.0% |
| Water | 87.8% |
| Carbomer | 0.1% |
| NaOH | 0.01% |
| Squalane | 0.1% |
| Beeswax | 0.5% |
| Glyceryl Stearate | 0.5% |
| BG | 4.0% |
| Citrate | 0.01% |

### Example 6

### Body Soap Formulation

The aluminum hydroxide particles prepared in accordance with Example 1, except kaolin was used in place of TiO₂, were used in a body soap formulation having the following components:

| Ingredient | Amount |
|---|---|
| Aluminum hydroxide with kaolin | 10.0% |
| Water | 64.6% |
| Magnesium Methyl Cocoyl Taurate | 10.0% |
| Polyquaternium-10 | 0.4% |
| COCAMIDOPROPYL BETAINE | 7.5% |
| SODIUM C14-16 OLEFIN SULFONATE | 7.5% |

While the embodiments have been described with reference to specific examples and features, persons having ordinary skill in the art will appreciate that various modifications may be made to the embodiments without departing from the spirit and scope thereof.

What is claimed is:

1. A cosmetic composition comprising powder-containing aluminum hydroxide particles coated with a powder in an amount within the range of from about 60% to about 150% powder by weight of the aluminum hydroxide and, a cosmetically acceptable carrier.

2. The cosmetic composition as claimed in claim 1, wherein the powder is selected from the group consisting of sericite, muscovite, biotite, lithia mica, synthetic mica, kaolionite, kaolin, nacrite, dekkite, halloysite, sillimanite, kyanite, magnesium silicates, talc, titanium dioxide, zinc dioxide, and mixtures thereof.

3. The cosmetic composition as claimed in claim 2, wherein the powder is selected from the group consisting of mica, kaolin, talc, titanium dioxide, and mixtures thereof.

4. The cosmetic compositions as claimed in claim 3, wherein the powder is titanium dioxide.

5. The cosmetic composition as claimed in claim 1, wherein the powder-containing aluminum hydroxide particles have an average particles within the range of from about 0.01 to about 50 μm.

6. The cosmetic composition as claimed in claim 5, wherein the powder-containing aluminum hydroxide particles have an average particles within the range of from 1 to about 35 μm.

7. The cosmetic composition as claimed in claim 1, wherein the powder-containing aluminum hydroxide particles are present in the composition in an amount within the range of from about 1 to about 65% by weight.

8. The cosmetic compositions as claimed in claim 7, wherein the powder-containing aluminum hydroxide particles are present in the composition in an amount within the range of from about 5 to about 50% by weight.

9. The cosmetic composition as claimed in claim 1, wherein the powder-containing aluminum hydroxide particles are coated with a powder in an amount within the range of from about 75% to about 125% of coating by weight of the aluminum hydroxide.

10. The cosmetic composition as claimed in claim 9, wherein the powder-containing aluminum hydroxide particles are coated with a powder in an amount within the range of from about 85% to about 100% of coating by weight of the aluminum hydroxide.

11. The cosmetic composition as claimed in claim 1, wherein the composition is selected from the group consisting of a foundations, lip stick, eye shadow, lotion, cream, concealer, blush, eyeliner, mascara, eyebrow liner, lipliner, sunscreen, deodorant, antiperspirant, body soap, and shower gel.

12. A method of making a powder-containing aluminum hydroxide particle coated with a powder in an amount within the range of from about 60% to about 150% powder by weight of the aluminum hydroxide, comprising:
a) preparing an aqueous sodium hydroxide (NaOH aq) solution;
b) adding a powder to the solution with high speed to homogenize the mixture to a homogenized powder mixture that is uniformly dispersed;
c) contacting the homogenized powder mixture with an aqueous aluminum sulfate (Al₂(SO₄)₃) solution to form a mixture containing a powder-containing aluminum hydroxide particles;
d) filtering the powder-containing aluminum hydroxide particles from the mixture; and
e) drying the powder-containing aluminum hydroxide particles.

13. The method as claimed in claim 12, wherein the homogenized mixture of b) is heated to a temperature within the range of from about 30 to about 75° C.

14. The method as claimed in claim 13, wherein the homogenized mixture of b) is heated to a temperature of about 50° C.

15. The method as claimed in claim 12, wherein contacting in c) is carried out by step-wise adding the aqueous aluminum sulfate solution until the pH of the mixture reaches a value within the range of from about 5.5 to about 8.5.

16. The method as claimed in claim 15, wherein contacting in c) is carried out by step-wise adding the aqueous aluminum sulfate solution until the pH of the mixture reaches a value of about 7.0.

17. The method as claimed in claim 12, wherein drying the powder-containing aluminum hydroxide particles is conducted at a temperature within the range of from about 75° C. to about 175° C.

18. The method as claimed in claim 17, wherein drying is carried out at a temperature of about 105° C.

19. The method as claimed in claim 12, wherein the powder is selected from the group consisting of sericite, muscovite, biotite, lithia mica, synthetic mica, kaolionite, kaolin, nacrite, dekkite, halloysite, sillimanite, kyanite, magnesium silicates, talc, titanium dioxide, zinc dioxide, and mixtures thereof.

20. The method as claimed in claim 19, wherein the powder is selected from the group consisting of mica, kaolin, talc, titanium dioxide, and mixtures thereof.
